(19) United States
(12) Patent Application Publication
(10) Pub. No.: US 2003/0138857 A1
(43) Pub. Date: Jul. 24, 2003

(54) Method of inhibiting atherosclerotic plaque destabilization

(76) Inventors: Pascal J. Goldschmidt-Clermont, Chapel Hill, NC (US); Dean J. Kereiakes, Cincinnati, OH (US); Puvi Sheshiah, Scottdale, MI (US)

Correspondence Address: NIXON & VANDERHYE, PC, 1100 N GLEBE ROAD, 8TH FLOOR, ARLINGTON, VA, 22201-4714, US

(21) Appl. No.: 10/342,330

(22) Filed: Jan. 15, 2003

Related U.S. Application Data
(60) Provisional application No. 60/347,877, filed on Jan. 15, 2002.

(51) Int. Cl. G01N 33/53; G01N 33/567; A61K 38/18

(52) U.S. Cl. 435/7.2; 514/12

(57) ABSTRACT

The present invention relates, in general, to atherosclerotic plaque rupture and, in particular to a method of identifying compounds suitable for use in inhibiting atherosclerotic plaque destabilization and to a method inhibiting plaque rupture and thrombosis using compounds so identified.

This application claims priority from Provisional Application No. 60/347,877, filed Jan. 15, 2002, the entire content of which is incorporated herein by reference.

### TECHNICAL FIELD

The present invention relates, in general, to atherosclerotic plaque rupture and, in particular, to a method of identifying compounds suitable for use in inhibiting atherosclerotic plaque destabilization and to a method inhibiting plaque rupture and thrombosis using compounds so identified.

### BACKGROUND

Atherosclerotic plaque rupture with superimposed thrombosis is the main cause of acute coronary syndromes (ACS). Therefore, to preserve event-free survival, the challenge is not simply to suppress atherogenesis but to suppress life-threatening plaque rupture and ulceration in existing plaques. Three major determinants of plaque rupture are the size and consistency of the atheromatous lipid core, fibrous cap thickness, and the intensity of inflammation and repair within the core (Zhou et al, Scand. J. Clin. Lab. Invest. Suppl. 230:3-11 (1999)). A lipid core occupying >40% of the plaque area (Lendon et al, Atherosclerosis 87:87-80 (1991)) and a thin, collagen-poor fibrous cap (Loree et al, Arterioscler. Thromb. 14:230-234 (1994)) increase the risk of plaque rupture and thrombosis. Caps of ruptured plaques are weakened when the macrophage density increases (Davies et al, Br. Heart J. 69:377-381 (1993)). Furthermore, macrophage-rich plaques are found more often in the coronary arteries of patients with unstable angina and non-Q-wave myocardial infarction than in those of patients with stable angina (Moreno et al, Circulation 90:775-778 (1994)).

Human leukocyte antigen-DR expression, a marker of macrophage activation, is found more often on macrophages of ruptured plaques (Arhustini et al, Heart 82:269-272 (1999), Farb et al, Circulation 93:1354-1363 (1996)). Moreover, fibrous caps that have ruptured not only have twice as many macrophages as unruptured fibrous caps but contain half as many smooth muscle cells, indicating that the ratio of vascular smooth muscle cells (VSMCs) to monocytes/macrophages (MMs) is altered markedly in ruptured plaques. The proportion of VSMCs undergoing apoptosis is increased significantly in unstable angina plaques versus stable angina (Bourjedel et al, Cardiovasc. Res. 41:480-488 (1999)).

Macrophage colony-stimulating factor (M-CSF) is a hematopoietic growth factor supporting survival, proliferation, and differentiation of monocytic cells. Recent studies suggest that M-CSF is the strongest predictor of ACS (Saitoh et al, J. Am. Coll. Cardiol. 35:655-665 (2000)). Both VSMCs and endothelial cells produce M-CSF when exposed to a variety of stimuli (Filonzi et al, Atherosclerosis 99:241-252 (1993), Zoellner et al, Blood 80:2805-2810 (1992)) Macrophages within atheromatous vessels seem to be the primary target for M-CSF (Clinton et al, Am. J. Pathol. 30:301-316 (1992)). M-CSF is also produced by macrophages themselves (Shyy et al, J. Clin. Invest. 92:1745-1751 (1993)) and on engagement of their FcγR receptors (Marsh et al, J. Immunol. 162:6217-6225 (1999)). Elevated C-reactive protein (CRP) levels have been shown to be associated with ACS (Anderson et al, J. Am. Coll. Cardiol. 32:35-41 (1998), Morrow et al, J. Am. Coll. Cardiol. 31:1460-1465 (1998)). CRP inflammatory process involving macrophages, T and B cells, immunoglobulin (Ig) G deposition, and M-CSF production. CRP has a proinflammatory effect on endothelial cells and VSMCs (Pasceri et al, Circulatrion 102:2165-2168 (2000)). M-CSF is affected by CRP and interleukin (IL)-1 β levels, suggesting a relation between atherogenic cytokines and acute-phase proteins in patients with ACS (Ikonomidis et al, Circulation 100:793-798 (1999)).

The present invention results from studies demonstrating that M-CSF is the common denominator to plaque destabilization and that M-CSF promotes VSMC killing by activating MMs, causing the consequent loss of VSMCs within vulnerable plaques. The present invention provides methods of identifying compounds that can be used to prevent plaque rupture and therapeutic strategies based on compounds so identified. The present invention also provides a method of detecting plaque rupture.

### SUMMARY OF THE INVENTION

The present invention relates generally to atherosclerotic plaque rupture. More specifically, the invention relates to a method of identifying compounds suitable for use in inhibiting atherosclerotic plaque destabilization and to a method inhibiting plaque rupture and thrombosis using compounds so identified.

Objects and advantages of the present invention will be clear from the description that follows.

### BRIEF DESCRIPTION OF THE DRAWINGS

FIGS. 1A-1C. (FIG. 1A). Overlay of fluorescent micrographs and (differential interference contrast) DIC images. Co-cultures were stained with Hoechst 33342 (360 nm) and propidium iodide (PI, 555 nm), for apoptosis. Light blue staining of intact nuclei is typical of healthy live cells. Cells with PI-positive nuclei, often pycnotic due to chromatin condensation, nuclei are undergoing apoptosis. Membrane permeability manifested by the pink coloration of the cytoplasm is indicative of late apoptosis. Bar=10 μm. (I) Control VSMCs; (II) VSMC with unstimulated monocytes; (III) VSMCs co-cultured with monocytes and M-CSF (100 ng/ml); (IV) (Inset in III), monocytes attached to apoptotic VSMCs which reveal nuclear condensation; (V) VSMCs with unstimulated monocytes; (VI) apoptotic VSMCs with monocytes attached showing morphological features of apoptosis. (FIG. 1B) VSMC apoptotic index is expressed as percent apoptotic cells to total VSMCs (mean percent±SEM). It is only when monocytes are stimulated with M-CSF (50 ng/ml) that significant VSMC death occurs. VSMCs were grown in serum free media, (*p≦0.0001). (FIG. 1C) Identical conditions as in FIG. 1B except that cells were grown in 5% serum supplemented media. (*p≦0.001).

FIG. 2. VSMCs pre-incubated with ZVAD fmk (10 μM/L) significantly decreased the activated monocyte-induced VSMC death (from 60±2.1% to 15.4±0.4%, P≦0.001).

FIGS. 3A and 3B. (FIG. 3A) Concentration-dependent effect of M-CSF on apoptosis of VSMCs exposed to monocytes (apoptotic index, mean±SEM). Half-maximal killing occurs at approximately 5 ng/ml of M-CSF. (FIG. 3B) M-CSF-activated monocytes induce apoptosis of VSMCs in the absence (*p≦0.001), but not in the presence of a 3 micron filter that separates the two cell types. Hence, direct contact between monocytes and VSMCs appears to be required to allow for enhanced apoptosis of VSMCs.

FIG. 4. M-CSF requirement for high concentration IL-1-mediated killing of VSMCs by MMs. IL-1 (100 pg/ml) did not induce significant killing of VSMCs (10.5±2.4%), relative to control VSMCS, in contrast with M-CSF activated monocytes (60.0±3.0%; *p≦0.005). Higher doses of IL-1 (500 and 1000 pg/ml) do cause activation of monocyte-induced VSMC death (59.5±3.5% and 59±3.8% respectively; **p≦0.01 compared to unstimulated monocytes). However, in the presence of IL-1 (500 and 1000 pg/ml), an anti-M-CSF (1 μg/ml) significantly blocked the VSMC death (17±2.1% and 13.6±1.9% respectively) in contrast to an isotype control (IgG2a, 1 μg/ml)) which was without effect (55.0±2.0%; ^{†}p≦0.001, monocytes with IL-1 alone compared to IL-1 and anti-MCSF); ^{††}p≦0.001, monocytes with IL-1 alone compared to IL-1 and anti-M-CSF).

FIG. 5. VSMC apoptosis in the presence of GPIIb/IIIa blockers. Abciximab (7 μg/ml) is able to inhibit M-CSF-triggered, monocyte-induced apoptosis of VSMCs (*p<0.0003). However, eptifibatide (5 μg/ml), and tirofiban (0.35 μg/ml) are unable to confer similar protection. VSMC apoptosis induced by M-CSF-activated monocytes was significantly blocked by an anti CD-18 antibody (3.5 μg/ml; 16.5±0.4%; **p≦0.00004). This inhibition was similar to that observed with Abciximab (19.1±2.2%).

FIG. 6: Comparison of the level of M-CSF and IL-6 in patients admitted to the catheterization laboratory.

### DETAILED DESCRIPTION OF THE INVENTION

The present invention is based, at least in part, on studies revealing the mechanism by which macrophage kill smooth muscle cells in the arterial plaque, through programmed cell death (apoptosis), that is triggered by the addition of M-CSF. The results provided in the Examples that follow demonstrate that M-CSF exposure is absolutely required for death induced by macrophages to occur. The death of smooth muscle cells induced by inflammatory cells is the determinant event in the rupture of atherosclerotic plaques.

The present invention provides a method of inhibiting the killing of smooth muscle cells and thereby preventing or minimizing plaque rupture. The method comprises administering an agent that blocks M-CSF activation of MMs in downstream molecular reactions. As plaque rupture represents the driving event for most strokes, heart attacks and other atherothrombotic events, the present method provides a highly significant therapy.

Agents suitable for use in the method of the invention include compounds (proteinaceous or non-proteinaceous) that target M-CSF and thereby inhibit the ability of M-CSF to activate macrophage-induced killing of smooth muscle cells. The present invention provides a method of screening test agents for their suitability for use in the instant method. The method can take the form, for example, of a binding assay. One such assay comprises contacting the agent to be tested with M-CSF (or portion thereof (e.g., the M-CSF receptor binding domain), or fusion protein comprising same) and with a binding target therefor (e.g., the M-CSF receptor or M-CSF binding portion thereof), and determining the effect of the test agent on the association of M-CSF (or portion thereof or fusion protein comprising same) with the binding target (e.g., by FACS).

Such assays can take the form of cell-free competition binding assays. For example, M-CSF or portion thereof or fusion protein containing same, can be incubated with the binding target, which binding target can bear a detectable label (e.g., a radioactive or fluorescent label). A test agent (proteinaceous or non-proteinaceous) can be added to the reaction and assayed for its ability to compete with the binding target for binding to M-CSF or portion thereof (or fusion protein). Free binding target can be separated from bound binding target, and the amount of bound target determined to assess the ability of the test agent to compete. This assay can be formatted so as to facilitate screening of large numbers of test agents, for example, by linking M-CSF or portion thereof (or fusion protein), to a solid support so that it can be readily washed free of unbound reactants. It will be appreciated that the binding target, rather than M-CSF, can be bound to a support and that either or both can bear a detectable label (e.g., a fluorescent or radioactive label) (advantageously, different labels when both are label-bearing), as can the test agent. The assays can also take the form of cell- or membrane-based assays.

M-CSF, or portion thereof (or fusion protein), suitable for use in assays such as that described above can be, as appropriate, isolated from natural sources or prepared recombinantly or chemically. M-CSF, or portion thereof, can be prepared as a fusion protein (e.g., a TNF fusion) using, for example, known recombinant techniques. The non M-CSF moiety can be present in the fusion protein N-terminal or C-terminal to the M-CSF, or portion thereof.

As indicated above, M-CSF or portion thereof or fusion protein, can be present linked to a solid support, including glass or plastic chips, slides or plates, agarose and nitrocellulose. Methods of attachment of proteins to such supports are well known in the art and include direct chemical attachment and attachment via a binding pair (e.g., biotin and avidin or biotin and streptavidin).

Agents identifiable using the above-described assay as being capable of modulating the association between M-CSF and the binding target can be further assayed (or assayed initially) for their ability to inhibit the ability of M-CSF to activate macrophage-induced killing of smooth muscle cells. For example, a mixed culture of human monocytes and smooth muscle cells can be used wherein killing of the smooth muscle cells is activated by macrophage triggered with M-CSF. The end point of such an assay is death of the smooth muscle cells by apoptosis. A test agent revealed as being capable of reducing apoptosis can be expected to be useful in inhibiting plaque rupture. Example 3 includes a description of one such assay that can be carried out in the presence and absence of test agent and the difference in the resulting cell death determined.

The invention encompasses agents identified or identifiable using the above-described assays. Such agents can include small molecules (e.g., organic compounds (for example, organic compounds less than about 500 Daltons)), and protein (e.g., antibodies (see Examples)) or soluble Fas (sFas) (CD95) (e.g., the extracellular domain of Fas (e.g., rat) encoded in base 56 to 565 (amino acid 28 may be serine rather than phenylalanine)), polypeptides, oligonucleotides, as well as natural products (preferably, in isolated form).

Agents of the invention can be formulated as pharmaceutical compositions comprising the agent(s) and a pharmaceutically acceptable diluent or carrier. The composition can be present in dosage unit form (e.g., as a tablet or capsule) or as a solution, preferably sterile, particularly when administration by injection is anticipated. The dose and dosage regimen will vary, for example, with the patient, the agent and the effect sought. Optimum doses and regimens can be determined readily by one skilled in the art.

Agents identified or identifiable using one or more of the above assays can be potentially used in the clinical management of strokes, heart attacks and other atherothrombotic events.

The present invention further relates to methods of identifying individuals at particular risk for heart attacks based on the measurement of M-CSF levels in the blood. The present invention further provides a method of identifying unstable plaques, that is, those associated with large quantities of M-CSF. Magnetic resonance imaging or VET scanning can be used to image such plaques.

The invention additionally relates to kits, for example, kits suitable for conducting screens/assays described herein. Such kits can include M-CSF or portion thereof or fusion protein comprising same, and/or binding target, free or bound to a support. One or more of these components can bear a detectable label. The kit can include any of the above components disposed within one or more container means. The kit can further include ancillary reagents (e.g., buffers) for use in the screens/assays.

Certain aspects of the invention are described in greater detail in the non-limiting Examples that follows.

### EXAMPLE 1

Experimental Details

Reagents

Reagents were obtained from the following sources: recombinant M-CSF, anti-M-CSF mAb, clone-26730.11, and IL-1 β (R&D Systems); abciximab (ReoPro), eptifibatide, and aggrastat (hospital stores); cell culture media and supplements (Gibco-BRL and Clonetics); lymphoprep (Nycomed); anti-CD-14-FITC, clone-M5E2, and anti-CD-45-PE, clone-HI30, antibodies (Pharmingen); anti-α-smooth muscle actin (Sigma Chemicals); human monocyte isolation kit (Miltenyi Biotec); and pan-caspase inhibitor (ZVAD-fmk) (Calbiochem). Anti-CD-18 antibody was a gift from Hal Baron and Eugen Koren of Genentech.

Smooth Muscle Cell Culture

VSMCs (passage 3 to 7) were obtained from Clonetics and from heart donors. VSMC identity was confirmed by α-smooth muscle actin staining. VSMCs initially were grown in growth medium (SmGM2; Clonetics) with 5% serum, and at 60% to 70% confluence, the media was changed to a serum-free medium (50:50 of DMEM/F12 media with 5 mL of ITS, PSA, L-glutamine, and nonessential amino acids per 500 mL of solution). After 72-hour incubation in the serum-free medium, VSMCs were co-cultured with monocytes. Co-culture experiments were also conducted with the use of SmGM2 media (5% serum) throughout the experiment.

Monocyte Preparation

Leukocytes were isolated from buffy coat preparations (American Red Cross) by means of the Ficoll-Paque gradient method. Monocytes were isolated by means of an indirect magnetic labeling system and a monocyte isolation kit. The purity of monocytes obtained was 90±64%, which was confirmed by flow cytometry with anti-CD-14-FITC and anti-CD45-PE antibodies.

Microscopy

Fluorescence microscopy (magnification ×400 and ×600) was performed with a Nikon Eclipse 800 microscope fitted with Namarski optics for differential interference contrast and an epifluorescence attachment. Excitation filters used were 360 nm, 490 nm, and 555 nm. For phase contrast microscopy, a Nikon TMS inverted phase contrast microscope was used. Digital images from the fluorescent microscope were captured with a Photometrics CCD camera.

Co-Culture Experiments

Freshly isolated monocytes were added 3:1 to VSMCs. Conditions included wells with VSMCs alone (with or without M-CSF) and VSMCs co-cultured with monocytes (with and without M-CSF). Experiments were conducted with either no serum or 5% serum in media. Experiments were also repeated by preincubating VSMCs with a pan-caspase inhibitor (1 hour at 37° C.) (ZVAD-fmk [10 μmol/L/mL]; sequence Z-Val-Ala-Asp [Ome]-CH₂ F), before addition of the MMs and M-CSF. Media and ZVAD-fmk were replenished every 24 hours. Specific controls were used for VSMCs with ZVAD-fmk.

Abciximab, aggrastat, and eptifibatide, 3 established parenteral glycoprotein (GP) IIb/IIIa blockers, were added to MM and VSMC co-cultures before exposure to M-CSF. Controls were used for VSMCs with abciximab, aggrastat, and eptifibatide separately, with or without M-CSF. All experiments were done in triplicate.

Experiments were done with the use of recombinant human IL-1 β (100 to 1000 pg/mL) in place of M-CSF. Anti-human M-CSF neutralizing antibody (1 μg/mL) was added along with IL-1 β, and the VSMCs plus monocytes were co-cultured for 72 hours. Isotype control, IgG2a (clone 20102.1), was used for VSMCs cultured with monocytes and IL-1 β.

Apoptosis Assay

After 72-hour incubation, MM and VSMC co-cultures were stained live with the Vybrant Apoptosis kit (Molecular Probes), consisting of 2 nuclear stains, Hoechst 33342 (5 mg/mL) and propidium iodide (1 mg/mL). After incubation for 20 minutes at 37° C., coverslips were washed and the cell death index was estimated as previously described (Webster et al, J. Clin. Invest. 104:239-252 (19999)) (apoptotic index=dead cells/[live cells+dead cells]×100).

Statistical Analysis

Statistical analysis was performed with Systat 9 software from SPSS Software, Inc, and Prism 3.0 from Graphpad, Inc. Data were analyzed by ANOVA, and the Bonferroni multiple comparison post test was used to compare pairs of group mean±SEM of individual experimental groups (groups compared are specified in each experiment). For statistical significance, a value of P≦0.05 was considered significant.

Results

Monocytes induce VSMC apoptosis when activated with M-CSF

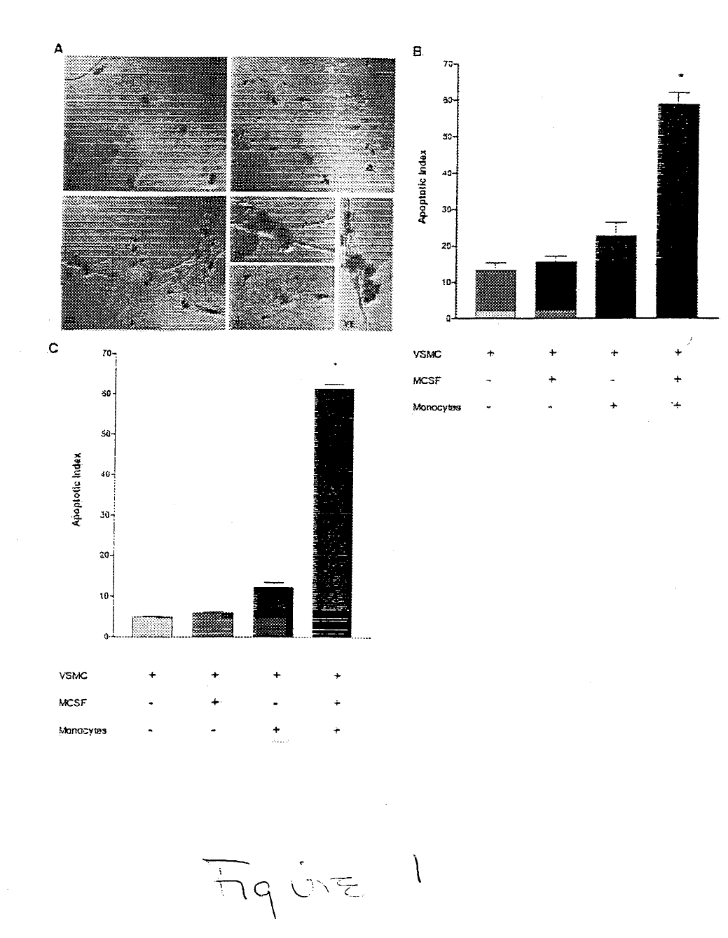

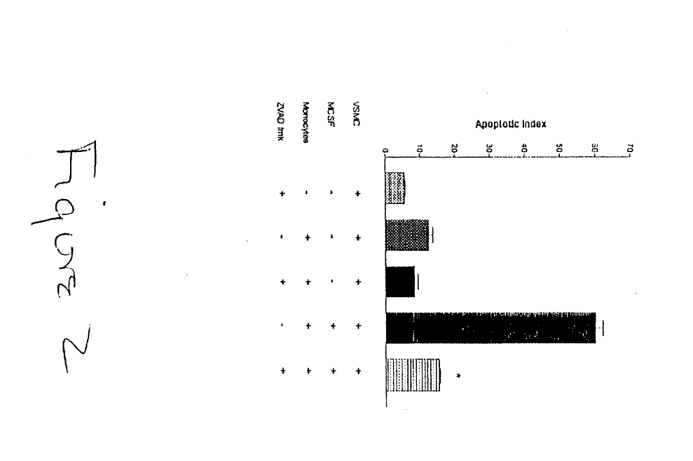

The apoptosis rate of VSMCs exposed to both monocytes and M-CSF is increased markedly (results from 4 sets of experiments). Thus, the apoptotic index was 13.2±2.1% for control VSMCs, 15.7±1.5% for VSMCs cultured with M-CSF (100 ng/mL) but without monocytes, and 22.7±3.7% for VSMCs co-cultured with monocytes without M-CSF. In contrast, VSMCs cultured with both M-CSF and monocytes display a markedly increased apoptotic index (58.8±3.3%). The differences in apoptotic indexes between VSMCs cultured with monocytes and M-CSF versus all control conditions are significant (P<0.0001) (FIG. 1B). Similar results were obtained when VSMCs were grown in serum-supplemented media (5% serum), although the basal indexes of apoptosis were lower than in the absence of serum (FIG. 1C).

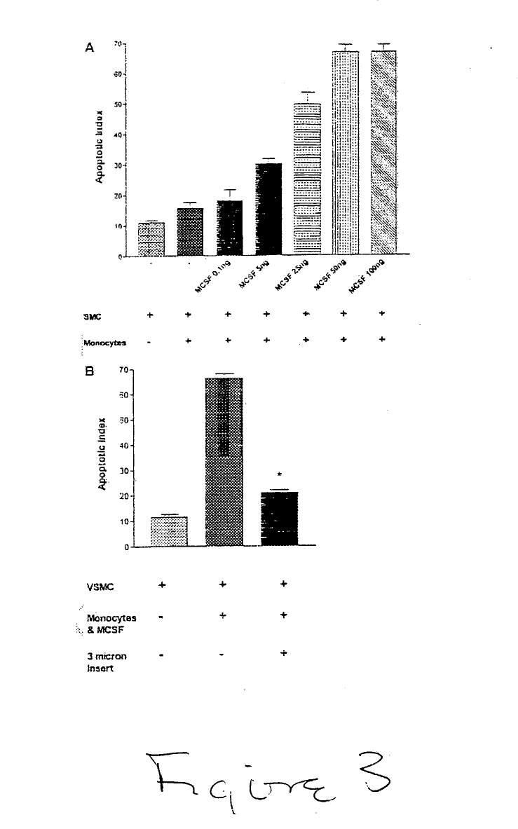

It was confirmed that the death process was through apoptosis with the use of a pan-caspase inhibitor, ZVAD-fmk. When VSMCs were preincubated with ZVAD-fmk (10 μmol/L/mL), apoptosis induced by co-culture with activated monocytes was completely abrogated. Thus, VSMCs cultured with monocytes and M-CSF exhibited an apoptotic index of 60.0±2.1%, and the pan-caspase inhibitor ZVAD-fmk decreased this index to 15.4±0.4% (P≦0.001), similar to that observed in smooth muscle cells cultured with monocytes alone (12.2±1.6%) (FIG. 2).

Effect of M-CSF is Concentration-Dependent and Requires Direct Contact Between Monocytes and Smooth Muscle Cells

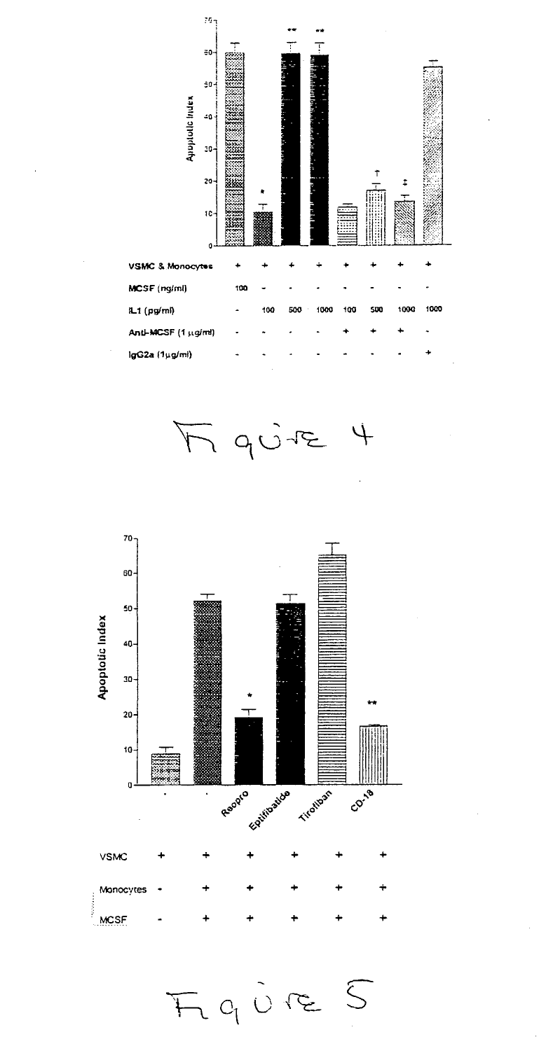

Concentrations of M-CSF ranging from 0.1 ng/mL to 100 ng/mL were tested. Maximum VSMC apoptosis was obtained with 50 ng/mL and 100 ng/mL, whereas half-maximal killing occurs at a physiological dose of 5 ng/mL (FIG. 3). The data are consistent with a process in which MMs, on engagement of their M-CSF receptor, can trigger the cell death program of VSMCs. Next, a determination was made as to whether activation of the cell death program of VSMCs required physical contact between VSMCs and activated MMs. Monocytes were separated from the VSMCs with a porous insert (3-μm-diameter pores), and when stimulated with M-CSF (100 ng/mL) were unable to induce VSMC apoptosis (21.0±1.0%) compared with VSMCs co-cultured and in direct contact with activated monocytes (66.3±1.8%) (P≦0.001) (FIG. 3).

Activation of Monocyte Killing Activity Toward VSMCs is Specific to M-CSF

To test if the activation of the observed monocyte-induced killing is specific to M-CSF, another common monocyte-activating cytokine, IL-1, were studied. IL-1 is known to induce monocyte adherence. Co-culture experiments were repeated by using IL-1 instead of M-CSF. IL-1 at physiological concentrations (100 pg/mL) did not induce monocyte-induced VSMC apoptosis. VSMCs cultured with monocytes and IL-1 (100 pg/mL) exhibited an apoptotic index of 10.5±2.4%, which was similar to that of control VSMCs and VSMCs cultured with unactivated monocytes, whereas M-CSF activation of monocytes significantly increased VSMC apoptosis (60.0±3.0%).

Markedly higher doses of IL-1 (500 pg/mL and 1 ng/mL) did cause increased monocyte-induced killing of VSMCs (59.5±3.5% and 59.0±3.8%, respectively) (FIG. 4). However, monocyte killing of VSMCs in response to high doses of IL-1 was dependent on the endogenous production of M-CSF induced by IL-1. Thus, when VSMC killing by monocytes was triggered by IL-1 (500 pg/mL and 1000 pg/mL), such killing was completely abrogated in the presence of 1 μg/mL of anti-M-CSF-blocking antibody (17.0±2.1% and 13.6±1.9%, respectively; P≦0.001 and P≦0.001, respectively) (FIG. 4). A nonspecific isotype-matched control antibody, IgG2a (1 mg/mL), was used as control and did not block IL-1-induced (1000 pg/mL) VSMC apoptosis (55.0±2.0%) (FIG. 4). These experiments, taken together, indicate that M-CSF functions as a rate-limiting cytokine in the process of monocyte killing of VSMCs. IL-1, even at physiological levels, has been shown to induce the production M-CSF by VSMCs and monocytes (Filonzi et al, Atherosclerosis 99:241-252 (1993), Zoellner et al, Blood 80:2805-2810 (1992), (Pasceri et al, Circulation 102:2165-2168 (2000)).

Chimeric Monovalent Antibody that Cross-Reacts with Mac-1, Abciximab, Blocks M-CSF-Induced Monocyte Killing of VSMCs

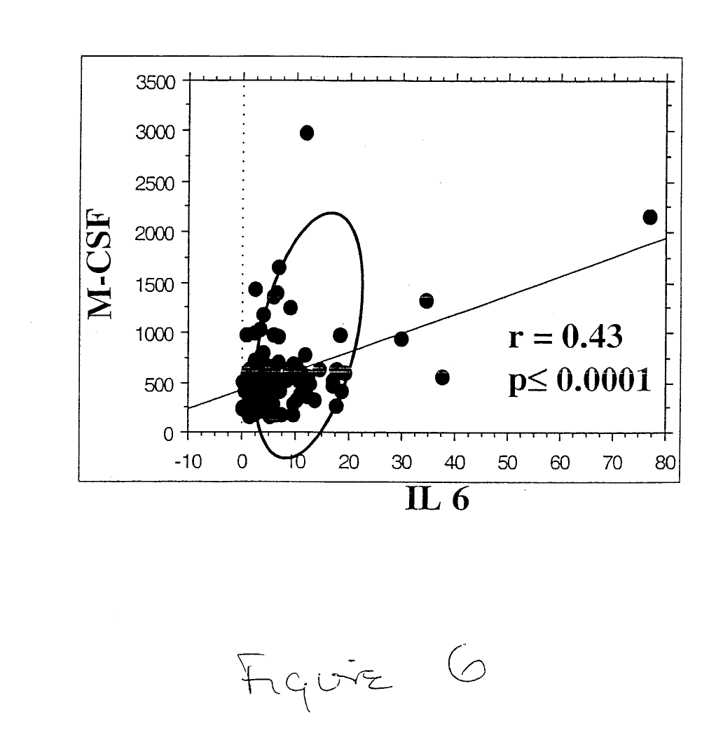

Engagement of the α_{M}β₂ receptor (CD11b/CD18 or Mac-1) plays a central role in multiple activities of macrophages and granulocytes in inflammatory processes, including atherosclerosis. The chimeric monovalent and humanized monoclonal antibody Fab fragment abciximab binds with high affinity to the activated conformation of Mac-1 (Plescia et al, J. Biol. Chem. 273:20372-20377 (1998)). Abciximab at therapeutic concentration (7 μg/mL) was added to VSMCs alone or to co-cultures of VSMCs and monocytes, with and without M-CSF. A significant decrease in the apoptotic index of VSMCs occurs when abciximab is added to VSMCs co-cultured with monocytes in the presence of M-CSF (P<0.0003, relative to identical conditions except for the absence of abciximab) (FIG. 5). Abciximab also binds α_{IIb}β₃ (GP IIb/IIIa or fibrinogen receptor) and the activated conformation of the α_{V}β₃ (the vitronectin receptor) (Tam et al, Circulation 98:1085-1091 (1998)). Therefore, the possibility was tested that other GP IIb/IIIa blockers, such as eptifibatide and tirofiban, also could reduce the pro-apoptotic effect of M-CSF-activated macrophages on VSMCs. These inhibitors are highly efficient at blocking platelet aggregation but, unlike abciximab, do not interfere with Mac-1 binding (Plescia et al, J. Biol. Chem. 273:20372-20377 (1998)). Neither tirofiban nor eptifibatide detectably inhibited the M-CSF-induced monocyte killing of VSMCs. The cell death index in the presence of tirofiban (therapeutic concentration, 0.35 μg/mL) was 65.0±3.4% and for eptifibatide (therapeutic concentration, 5 μg/mL) was 51.3±2.2% (FIG. 5), not significantly different from that of VSMCs exposed to M-CSF-activated MMs (51.9±1.6%, P=0.76). However, the difference between the apoptotic indexes obtained in the presence of tirofiban or eptifibatide versus abciximab (19.1±2.3%) was significant (P≦0.0006 and P≦0.001, respectively) (FIG. 5). The contribution of Mac-1 engagement for monocyte-induced killing of VSMCs was further tested by using a human recombinant anti-CD-18 antibody. This anti-CD-18 antibody at a dose of 3.5 μg/mL was able to inhibit M-CSF-induced monocyte killing (16.5±0.4%) when compared with M-CSF-activated monocytes co-cultured with VSMCs in the absence of anti-CD-18 (51.9±1.8%) (P≦0.00004). This inhibition was similar to that observed with abciximab (19.1±2.2%) (FIG. 5). These results obtained with a recombinant anti-CD-18 antibody further support the requirement of Mac-1-mediated cell-to-cell contact between M-CSF-activated monocytes and VSMCs to allow for execution of the death pathway that results in VSMC killing.

### EXAMPLE 2

IL-6 level has been shown to be a very good predictor for mortality in patients with unstable coronary syndromes (Lindmark et al, JAMA. 286:2107-13 (2001)). A comparison was made of the level of M-CSF and IL-6 in patients admitted to the catheterization laborabory. There was a strong correlation between elevated IL-6 levels and elevated M-CSF (see FIG. 6). Yet, there was a group of patients whose M-CSF level was elevated even in the absence of IL-6. The direct contribution of M-CSF to plaque destabilization, unlike many other markers, would explain the unique capacity of this molecule to identify patients at highest risk for a poor outcome (Saitoh et al, J Am Coll Cardiol. 35:655-65 (2000)).

### EXAMPLE 3

SMC are grown on T-75 flasks (Fisher Scientific Corp.) in growth medium (SmGM2-Clonetics) with 5% serum and subsequently seeded onto 6-well plates (Fisher). Autoclaved cover slips are placed at the bottom of each well of the 6-well plates before seeding the plates. Once 60% confluence is achieved, co-culture experiments with monocytes are started. Monocytes are isolated from peripheral blood from healthy donors as well as from buffy coat preparations obtained from the American Red Cross using Optiprep gradient centrifugation. The Optiprep stock is buffered with HEPES 1M solution. (0.1 ml of 1M HEPES+10 ml Optiprep). The leukocyte-rich preparation (LRP) is mixed with Optiprep (2:0.8, vol:vol) to adjust the Optiprep to 1.1 g/ml. Two additional Optiprep solutions were mixed—Solution A (1.068 g/ml) by mixing 1 vol. of Optiprep with 4 vol. RPMI containing 10% fetal calf serum and Solution B (1.078 g/ml) by mixing 1 vol. of Optiprep with 3 vol. of RPMI medium containing 10% fetal calf serum. LRP (2 ml) is added to 15 ml—polypropylene centrifuge tube (Fisher Scientific) and centrifuged at 600×g for 10 min. Then 2 ml of Solution B is carefully layered on top of the LRP layer followed by 4 ml of Solution A and then finally a 0.5 ml layer of RPMI. Tubes were centrifuged at 800×g for 15 min at 20° C. Monocytes accumulate as a cloudy layer just beneath the RPMI layer and are carefully aspirated and transferred to a 50 ml polypropylene tube. Monocytes are pooled and diluted with RPMI (twice the volume). This monocyte—rich mixture is centrifuged at 1000×g for 5 min and then monocytes are re-suspended in fresh RPMI. The cells are counted with a hemocytometer and polymyxin B (10 μg/ml) is added to reduce endotoxin activity. The resulting monocyte fraction is 75%±6% pure using CD-14 fluorescent antibody as a marker (Pharmingen—Clone M5E2 FITC). Once the VSMCs are grown to about 60-70% confluence on coverslips layed in six well plates, they are rinsed and fresh media is added. The number of SMC is estimated after detaching SMC with trypsin from a representative well, and counting the cells (in hemocytometer). Freshly isolated monocytes are then added to the other wells in a ratio of 3:1 to SMC. The experimental conditions include wells that have VSMC alone, with or without M-CSF, and SMC co-cultured with monocytes, with and without M-CSF. MCSF is added to wells that contain SMC and monocytes at the time of co-culture. After a 72-hr incubation, monocytes/macrophages-SMC co-cultures are washed twice with PBS and stained live with the Vybrant Apoptosis assay [Molecular Probes, Eugene, OR]. The assay kit consists of two nuclear stains, Hoechst 33342 and Propidium Iodide. After washing the coverslips twice with PBS, the wells are refilled with 1 ml of PBS, 1 ml of 5 mg/ml solution of Hoechst 33342 and 1 ml of PI. After incubation for 20 minutes at 37° C., the coverslips are washed twice with PBS and then mounted on slides with fluoromount and sealed. Cell death index is then estimated as described in Example 1.

All documents cited above are hereby incorporated in their entirety by reference.

What is claimed is:

1. A method of screening a test agent for the ability to modulate macrophage colony-stimulating factor (M-CSF) activation of macrophage-induced killing of smooth muscle cells comprising contacting M-CSF, or portion thereof, with a binding target therefor in the presence and absence of said test agent, and determining the effect of said test agent on the association of M-CSF, or portion thereof, with said binding target,
wherein a test agent that enhances or inhibits said association is a candidate modulator of said M-CSF activation.

2. The method according to claim 1 wherein said portion is a M-CSF receptor binding domain.

3. The method according to claim 1 wherein said M-CSF, or portion thereof, or said binding target, is present in a fusion protein.

4. The method according to claim 1 wherein said binding target is an M-CSF receptor or M-CSF binding portion thereof.

5. The method according to claim 1 wherein said contacting is effected in a cell-free system.

6. The method according to claim 1 wherein said contacting is effected in a cell-containing system.

7. The method according to claim 1 wherein at least one of said M-CSF, or portion thereof, said binding target, and said test agent bears a detectable label.

8. The method according to claim 1 wherein at least one of said M-CSF, or portion thereof, said binding target, and said test agent is bound to a solid support.

9. A method of modulating death of smooth muscle cells comprising administering to a patient in need of said modulation an agent identifiable by the method of claim 1 as capable of enhancing or inhibiting the association of M-CSF, or portion thereof, with said binding target, in an amount sufficient to effect said modulation.

10. A method of inhibiting plaque rupture comprising administering to a patient in need thereof an amount of an agent that inhibits M-CSF activation of macrophage-induced killing of smooth muscle cells sufficient to effect said inhibition of plaque rupture.

11. The method according to claim 10 wherein said agent is a protein.

12. The method according to claim 11 wherein said protein is an antibody or soluble Fas (sFas) (CD95).

13. The method according to claim 12 wherein said sFas comprises the extracellular domain of Fas.

14. The method according to claim 10 wherein said patient is at risk of a stroke or heart attack.

15. A method of identifying an individual at risk of suffering an atherothrombotic event comprising determining the M-CSF level in the blood of said individual, wherein an elevated level of M-CSF indicates that said individual is at said risk.

16. A method of inhibiting atherosclerotic plaque destabilization comprising administering to a patient in need thereof an amount of an agent that inhibits M-CSF activation of macrophage-induced killing of smooth muscle cells sufficient to effect said inhibition of atherosclerotic plaque destabilization.

17. A method of inhibiting thrombosis comprising administering to a patient in need thereof an amount of an agent that inhibits M-CSF activation of macrophage-induced killing of smooth muscle cells sufficient to effect said inhibition of thrombosis.
